(19) United States
(12) Patent Application Publication
(10) Pub. No.: US 2008/0190437 A1
(43) Pub. Date: Aug. 14, 2008

(54) Dental Orthosis

(76) Inventor: Michele Hervy-Auboiron, Paris (FR)

Correspondence Address: CANTOR COLBURN, LLP, 20 Church Street, 22nd Floor, Hartford, CT, 06103, US

(21) Appl. No.: 11/908,171

(22) PCT Filed: Mar. 7, 2006
(86) PCT No.: PCT/FR06/00506

§ 371 Date: Sep. 10, 2007

(30) Foreign Application Priority Data
Mar. 9, 2005 (FR) 05/02346

(51) Int. Cl. A61C 7/08; A61F 5/56

(52) U.S. Cl. 128/848; 433/6

(57) ABSTRACT

The invention concerns a dental orthosis (1) comprising upper co-operating means (2) adapted to co-operate with the upper jaw (4) and lower co-operating means (5) adapted to co-operate with the lower jaw (5), characterized in that said orthosis (1) includes means (16) for inducing a mandibular advance. More precisely, said lower co-operating means (3) for exerting pressure (16) on the chin bones of said lower jaw (5).

### TECHNICAL FIELD OF THE INVENTION

The present invention concerns a dental orthosis, intended particularly for the therapeutic treatment of obstructive sleep apnea syndrome (OSAS).

### BRIEF DESCRIPTION OF RELATED ART

OSAS is a disorder defined by a series of episodes of total or partial obstruction, leading to total or partial cessation of breathing during sleep, and more precisely either to hypopnea, that is to say a reduction in air flow, or to apnea, that is to say a cessation of air flow or of ventilation.

Apnea is generally defined as a cessation of air flow lasting for 10 seconds or more, and hypopnea is generally defined as a reduction in air flow of at least 50% for at least 10 seconds.

The main symptoms associated with OSAS, and therefore with apnea and/or hypopnea, are hypersomnolence, memory problems, difficulty in concentrating, behavioral problems, depressive syndromes, loss of libido, automatic behavior, and risk of road accidents.

OSAS is also linked to a high cardiovascular mortality rate, since it may cause left and/or right ventricular insufficiency, cardiac dysrhythmia, cerebrovascular accidents, systemic and/or pulmonary arterial hypertension, and ischemic heart disease.

Hypopnea is also accompanied by brief periods of waking that disrupt the individual's sleep and cause a fragmentation of sleep patterns, such that sleep is no longer restorative because of these interruptions in air flow and the resulting episodes of hypoxemia, hence a feeling of tiredness in the patient.

To remedy OSAS, a first approach is to consider dietary measures, that is to say weight loss and a more balanced intake of food, these measures generally being incomplete and shortlived.

Artificial respirators are known from the prior art, in particular ventilators operating with continuous positive pressure, comprising a mask that the user has to place on his face before going to sleep, this respirator providing the patient with air when he is in respiratory distress.

However, this device is relatively cumbersome, awkward to use and loud, leading to noise disturbances that are difficult for those immediately around the user to cope with.

Moreover, treatment with continuous positive pressure is not always accepted by the patient, because of the inconvenience of wearing a mask and because of the associated noise problems.

Pharmacological treatments are also available, but their efficacy is unpredictable and remains very limited.

Surgical procedures aimed at rectifying the organic defects that can cause OSAS are also conceivable, for example nasal surgery, surgery of the soft tissues (tonsillectomy, uvulopalatopharyngoplasty, pharyngotomy, basiglossectomy), or bone surgery, such as bimaxillary surgery, hyoid surgery, or mental transposition.

However, these operations are relatively complex and require hospitalization of the patient.

In most people suffering from OSAS, an important anatomical change occurs during sleep, particularly a change in the soft-tissue parts, namely the tongue, the soft palate, the pharyngeal walls and palatine tonsils.

This change also affects the craniofacial skeleton.

It has also been found that certain anatomical abnormalities are factors that promote OSAS, such as the presence of craniomaxillofacial abnormalities and retrognathism, for example the position of the jaw, the angle of the base of the cranium, and the height of the face.

The physiopathology of OSAS is not clearly defined and involves several factors, such as changes in the anatomy and in the resistance of the upper airways, and also in the control of the tone of the dilator muscles of the pharynx during sleep.

According to the prior art, dental prostheses have been proposed for mandibular propulsion in the context of treatment of OSAS, these prostheses being intended to be worn at night.

These dental prostheses are intended to prevent the patient from exerting muscle forces on the soft tissues of the upper airways, in particular the tongue, the pharynx, the soft palate, and the vocal cords.

Their aim in theory is to move the tongue forward in order thereby to free the posterior pharyngeal airway by a forced mandibular advancement associated with opening of the mouth.

To do this, mechanical thrust mechanisms in the form of connecting rods are used, or one-piece thrust mechanisms when the devices tension the suprahyoid and upper temporal muscle group.

However, the prostheses according to the prior art generate a viscoelastic muscle reaction of the mandibular retropulsers.

Although the viscoelastic reaction of the retropulsion muscles is responsible for the activator effect used in dentofacial orthopedics for correcting dento-alveolar dysmorphism, this mechanical effect is not sought in the treatment of OSAS and is likely to damage the dental/periodontal system and thus cause dental pain or pain in the temporomandibular joints.

These prostheses may therefore cause not inconsiderable side effects that should be avoided if the patients are to continue their treatment without suffering adverse effects.

Moreover, the muscle involvement in the mandibular advancement is forced, not induced.

### BRIEF SUMMARY OF THE INVENTION

The present invention aims to solve the problems of the prior art by means of a prosthesis that is easy to use, is of a simple structure, is easily adaptable, is easy to produce with simple elements, does not have the side effects associated with the prostheses according to the prior art (dental pain, for example), permits effective treatment of patients with OSAS with high percentage rates of success, and promotes the involvement of the muscles in the mandibular advancement.

In the context of treatment of OSAS, the present invention concerns a dental orthosis comprising upper cooperating means adapted to cooperate with the upper jaw and lower cooperating means adapted to cooperate with the lower jaw, characterized in that said orthosis includes means for inducing mandibular advancement, and more precisely the lower cooperating means comprise means for exerting pressure on the mental spines of said lower jaw.

In order to exert a relatively precise pressure and to bring about reflex mandibular advancement, the pressure means are in the form of at least one bracket with a rounded end covered by a plate made of a non-allergenic substance and produced from a steel wire.

To make the orthosis according to the invention easier to wear, the cooperating means are made of a non-allergenic substance, particularly of an acrylic resin.

h order to permit mandibular advancement, the lower cooperating means have means for controlling mandibular propulsion, composed of a thrust actuator of adjustable length.

The upper cooperating means advantageously comprise means for fixation to the upper jaw, composed of means for fixation to at least one molar, in the form of an Adams type hook.

### BRIEF DESCRIPTION OF THE DRAWINGS

The present invention is now described on the basis of purely descriptive examples, which do not in any way limit the scope of the present invention, and with reference to the attached illustrations, in which:

FIG. 1 shows a front view of the orthosis according to the invention placed in a patient's mouth;

FIG. 2 shows a side view of the orthosis according to the invention placed in the patient's mouth;

FIG. 3 shows a perspective view of the interior of a patient's mouth, with the orthosis according to the invention;

FIG. 4 shows a perspective front view of the orthosis according to the invention fixed on the upper jaw;

FIG. 5 shows a perspective side view of the orthosis according to the invention fixed on the upper jaw;

FIG. 6 shows a perspective three-quarter view of the orthosis according to the invention fixed on the upper jaw.

### DETAILED DESCRIPTION OF THE DRAWINGS

Throughout the description, the same elements are designated by the same reference numbers.

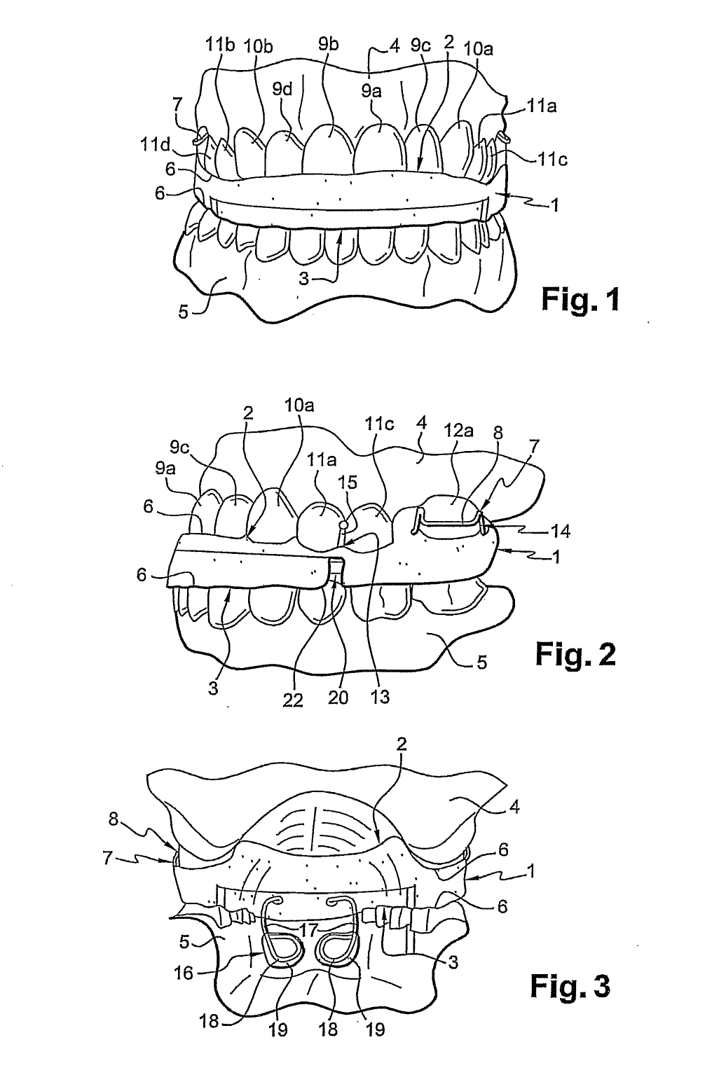

FIG. 1 shows a perspective front view of the orthosis according to the invention placed in a patient's mouth.

The orthosis 1 according to the invention has upper cooperating means 2 and lower cooperating means 3 that are adapted to cooperate with the upper jaw 4 and lower jaw 5, respectively, of a patient.

More precisely, the upper and lower cooperating means 2, 3 are in the form of a tray 6 in which the teeth of the upper jaw 2 and of the lower jaw 3 are able to bear and to cooperate.

The cooperating means 2, 3 have the general shape of a semicircle or horseshoe.

FIG. 1 shows cooperating means 2, 3 with an open outer face, that is to say that when the teeth are bearing on the tray 6, the outer face of the teeth is not covered by the cooperating means 2, 3, or is only partially covered.

It is nevertheless within the scope of the invention to produce cooperating means 2, 3 with a tray 6 that does cover the outer face of the teeth.

Moreover, the upper and lower cooperating means 2, 3 have hollows or internal cuttings of a shape matching the crowns of the teeth of the upper jaw 4 and lower jaw 5, respectively, these hollows or cuttings being advantageously formed from casts of the upper jaw 4 and lower jaw 5.

In this way, the crowns of the teeth can engage totally or partially in the tray 6, thus allowing the cooperating means 2, 3 to be held in a fixed position on the dental arches.

It is obviously within the scope of a person skilled in the art to produce such cooperating means 2, 3.

In order to avoid allergic reactions, the cooperating means 2, 3 are made of a non-allergenic substance, in particular of acrylic resin.

However, any type of substance is conceivable for producing cooperating means 2, 3 according to the invention.

FIG. 2 shows a side view of the orthosis according to the invention placed in a patient's mouth.

To allow the orthosis 1 to be held immovably in the patient's mouth, means 7 for fixing the orthosis 1 on the upper jaw 4 are provided in the upper cooperating means 2.

More precisely, the fixing means 7 are in the form of hooks 8 cooperating with the patient's upper dental arch.

Each person's teeth are usually made up of two pairs of incisors (9a, 9b, 9c, 9d), one pair of canines (10a, 10b), two pairs of premolars (11a, 11b, 11c, 11d) and then molars (12a, 12b), this arrangement of the teeth applying both to the upper jaw 4 and to the lower jaw 5.

The fixing means 7 shown in FIG. 2 are composed of means 13 of fixing with the aid of at least one premolar and means 14 of fixing to at least one molar.

The means 14 of fixing to at least one molar are in the form of a hook 8, presently known as an Adams hook.

The Adams hook 8 is thus advantageously produced from a hard steel wire which encloses a molar 12a, 12b and whose two ends are embedded in the upper cooperating means 2 and more precisely in the resin composition from which these cooperating means 2 are made.

It is entirely possible for a person skilled in the art to modify the configuration of the hook 8 depending on the molar on which it is to be fixed.

The means 13 for fixing the orthosis 1 with the aid of at least one premolar are in the form of an arm 15 advantageously made from an upright steel wire and able to be inserted between the two premolars 11a, 11c of the upper jaw 4.

The free end of the arm 15 advantageously has a ball or a bulged area of material in order not to nick or irritate the gums in the free space between the two premolars 11a, 11b, 11c, 11d.

A person skilled in the art will be able to adapt the length and diameter of the arm 15 depending on the size of the premolars 11a, 11b, 11c, 11d and of the free space between them.

FIG. 3 shows a perspective view of the interior of a mouth fitted with the orthosis according to the invention.

The presence of pressure means 16 in the area of the lower cooperating means 3 will be noted in FIG. 3, these pressure means 16 advantageously being able to exert a pressure on the mental spines of the patient.

The pressure means 16 are in the form of two brackets 17 with a rounded end 18 covered by a plate 19 made of a non-allergenic substance, for example an acrylic resin.

The plates 19 of the pressure means 16 are advantageously placed opposite the mucous zone covering the mental spines.

More precisely, the plates 19 are designed and configured in such a way that they exert a pressure on the genial tubercles.

The brackets 17 are advantageously produced from a steel wire presently used in the field of the invention and of a strength and rigidity sufficient to exert a pressure on the mucous zone.

Alternatively, a single bracket can be formed that is also able to exert a pressure on the mental spines.

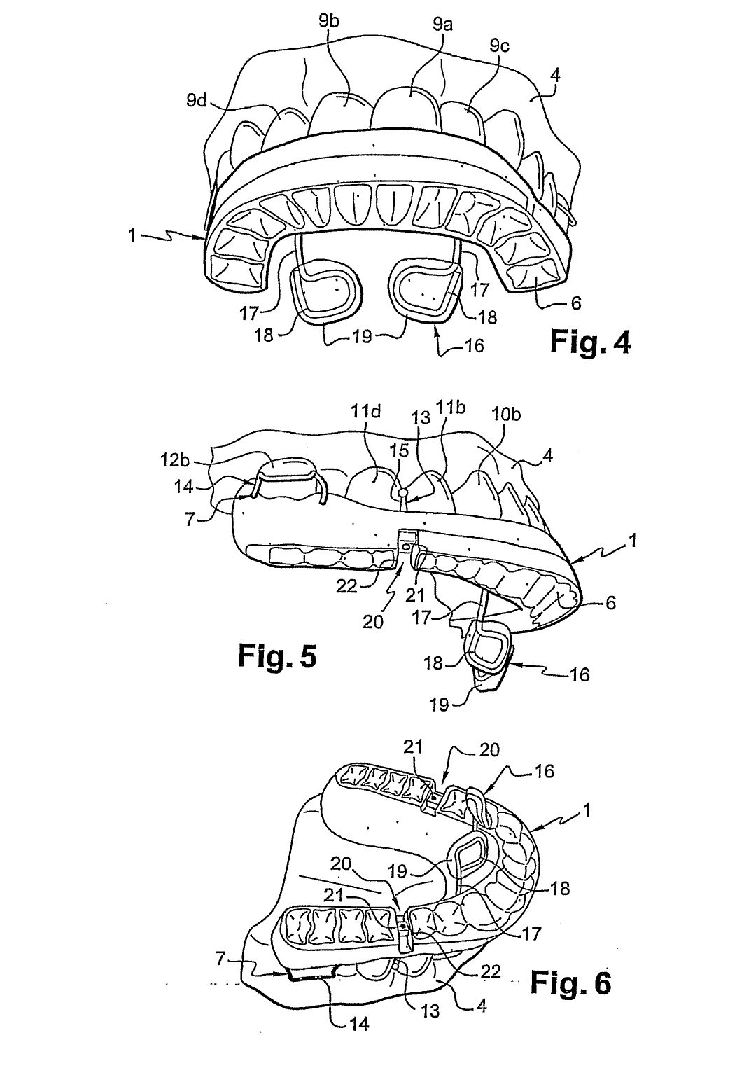

FIGS. 4 and 5 show a perspective front view and side view, respectively, of the orthosis according to the invention fixed on the upper jaw 4.

It will be seen from FIGS. 4 and 5 that the rounded end 18 of the brackets 17 is designed in the form of a loop or closed hook.

The stimulation deriving from the pressure exerted on the mental spines is transmitted to the central nervous system, which transforms it into efferent motor messages.

Mandibular advancement of the lower jaw 5 is stimulated and induced in this way.

FIG. 6 shows a third perspective view of the orthosis according to the invention fixed on the upper jaw 4.

To permit mandibular advancement of the lower jaw 5, the lower cooperating means 3 include means 20 for controlling the mandibular propulsion of the lower jaw 5, as can be seen in more detail in FIGS. 5 and 6.

When a person seeks to close his jaws together, he places the upper dental arch on the lower dental arch, and these engage on one another.

To obtain mandibular advancement, the lower cooperating means 5 are designed in such a way that the hollows or cuttings in the tray 6 for the crowns of the lower jaw 5 are placed in such a way that there is a mandibular advancement of at least a few millimeters relative to the occlusion position, this mandibular advancement being controlled by the means 20 for controlling mandibular propulsion.

Given the reception of the efferent motor messages deriving from the stimulation of the mental spines, no viscoelastic muscle reaction is generated, and the mandibular advancement is induced by a reflex reaction, not forced.

The means 20 for controlling mandibular propulsion are in the form of a thrust actuator 21 of adjustable length, thus making it possible to control the mandibular advancement.

More precisely, in order to customize and control the orthosis 1 as precisely as possible, it is necessary first of all to determine what mandibular advancement is induced by the patient when pressure is exerted on the mental spines.

Once the advancement obtained relative to the occlusion position has been determined, it is possible to adjust the length of the thrust actuator 21 accordingly and to form the orthosis with brackets 17 acting on the mental spines.

More precisely, the means 20 for controlling mandibular propulsion are provided symmetrically in the lower cooperating means 3 in the area of the right and left premolars or molars, respectively, of the lower jaw 5.

The means 20 for controlling mandibular propulsion are thus in the form of a material gap 22 in the lower cooperating means 3, and consequently in the tray 6.

The mandibular control means 20 make it possible to progressively regulate the mandibular advancement as a function of the muscle response induced by the patient, and thereby to cause an advance movement of the tongue and, consequently, greater freedom of the upper airways.

It should be noted that this increase in the length of the thrust actuator 21 brings about an advance movement and deformation of the lower cooperating means 3.

However, it is conceivable to produce an orthosis according to the invention without the means 20 for controlling mandibular advancement, and by producing an orthosis directly with cooperating means 2, 3 that are offset relative to the occlusion position of the patient, that is to say with a mandibular advancement relative to the occlusion position.

The orthosis thus obtained permits induction of a mandibular advancement that is no longer forced compared to the known prostheses of the prior art, but uniquely induced, and the pressure exerted in the area of the mental spines brings about a reflex mandibular advancement.

The mandibular advancement is therefore induced by the pressure means 16.

The orthosis according to the invention is adapted in particular for the treatment of obstructive sleep apnea syndrome.

1. A dental orthosis comprising:
upper cooperating means adapted to cooperate with tan upper jaws and;
lower cooperating means adapted to cooperate with a lower jaw;
wherein said orthosis has means for inducing mandibular advancement.

2. The orthosis as claimed in claim 1, wherein said lower cooperating means comprise means for exerting pressure on mental spines of said lower jaw.

3. The orthosis as claimed in claim 2, wherein said pressure means are in a form of at least one bracket.

4. The orthosis as claimed in claim 3, wherein said at least one bracket has a rounded end covered by a plate made of a non-allergenic substance.

5. The orthosis as claimed in claim 3, wherein said at least one bracket is formed from a steel wire.

6. The orthosis as claimed in claim 1, wherein said upper and lower cooperating means are made of a non-allergenic substance, particularly of an acrylic resin.

7. The orthosis as claimed in claim 1, wherein said lower cooperating means have means for controlling mandibular propulsion.

8. The orthosis as claimed in claim 7, wherein said means for controlling mandibular propulsion are composed of a thrust actuator of adjustable length.

9. The orthosis as claimed in claim 1, wherein said upper cooperating means comprise means for fixation to the upper jaw.

10. The orthosis as claimed in claim 9, wherein said fixation means are composed of means for fixing to at least one molar, in the form of an Adams type hook.
